(19) United States
(12) Patent Application Publication
(10) Pub. No.: US 2025/0229206 A1
(43) Pub. Date: Jul. 17, 2025

(54) AIR CLEANING MODULE AND AIR CLEANING DEVICE

(71) Applicant: Samsung Electronics Co., Ltd., Suwon-si (KR)

(72) Inventors: Huiyoung KIM, Suwon-si (KR); Juwan PARK, Suwon-si (KR); Nakhyun KIM, Suwon-si (KR); Juyoung KIM, Suwon-si (KR); Changhyun PARK, Suwon-si (KR); Wonhee LEE, Suwon-si (KR); Seoyoung CHO, Suwon-si (KR); Sunghyun CHUN, Suwon-si (KR)

(21) Appl. No.: 19/098,475

(22) Filed: Apr. 2, 2025

Related U.S. Application Data
(63) Continuation of application No. PCT/KR2023/013516, filed on Sep. 8, 2023.

(30) Foreign Application Priority Data
Oct. 6, 2022 (KR) 10-2022-0128133
Dec. 21, 2022 (KR) 10-2022-0180887

(51) Int. Cl. B01D 46/00; A61L 9/20; B01D 46/44; B03C 3/011; B03C 3/019; B03C 3/36; B03C 3/82

(52) U.S. Cl. CPC B01D 46/0045; A61L 9/20; B01D 46/0002; B01D 46/0032; B01D 46/44; B03C 3/011; B03C 3/019; B03C 3/368; B03C 3/82; A61L 2209/12; A61L 2209/14; B01D 2265/028; B01D 2273/30; B01D 2279/65

(57) ABSTRACT

An air purification module according to an embodiment may include: a housing, a fan provided inside the housing and configured to generate a flow inside the housing, a main body frame provided inside the housing and configured to support the fan, a support assembly including a support disposed in a lower portion of the main body frame to support the main body frame and configured to provide an intake port through which air is taken into the inside of the housing, and a controller comprising circuitry disposed in the support assembly and configured to control the fan, wherein the support assembly includes a base region in which the controller is disposed, an edge region configured to support the main body frame to be positioned at a specified height from the base region, and a support frame including a plurality of ribs configured to connect the edge region and the base region so that the intake port is provided between the base region and the edge region, and a frame upper cover comprising a material different from a material of the support frame, disposed on an upper portion of the base region, and configured to protect the controller and guide a movement of air from the intake port toward the fan.

### CROSS-REFERENCE TO RELATED APPLICATIONS

This application is a continuation of International Application No. PCT/KR2023/013516 designating the United States, filed on Sep. 8, 2023, in the Korean Intellectual Property Receiving Office and claiming priority to Korean Patent Application Nos. 10-2022-0128133, filed on Oct. 6, 2022, and 10-2022-0180887, filed on Dec. 21, 2022, in the Korean Intellectual Property Office, the disclosures of each of which are incorporated by reference herein in their entireties.

### BACKGROUND

### Field

The disclosure relates to an air purification module, and an air purification device.

### Description of Related Art

Air purification devices are used to remove contaminants from air. Air purification devices may remove chemicals, such as bacteria, viruses, fungi, fine dust, and the like, which cause bad odors, from inhaled air.

Air purification devices may include an air purification module that collects particulates in the air.

The air purification module may include an intake port drawing in polluted air, an exhaust port discharging purified air, and a fan for generating airflow.

Air purification devices may include an air sterilization module that sterilizes microorganisms in the air.

The air sterilization module may sterilize microorganisms, such as germs, viruses, etc. in the air by emitting ultraviolet rays into the airflow.

### SUMMARY

An air sterilization module according to an example embodiment may include: a housing; a fan provided inside the housing and configured to generate a flow inside the housing; a main body frame provided inside the housing and configured to support the fan; a support assembly disposed in a lower portion of the main body frame including a support configured to support the main body frame and configured to provide an intake port through which air is taken into the inside of the housing; a controller, comprising circuitry, disposed in the support assembly and configured to control the fan; a support frame including a base region in which the controller is disposed; an edge region configured to support the main body frame to be positioned at a specified height from the base; a support frame including a plurality of ribs configured to connect the edge region and the base region so that the intake port is formed between the base region and the edge region; and a frame upper cover comprising a material different from a material of the support frame, disposed on an upper portion of the base region, configured to protect the controller, and to guide a movement of air from the intake port toward the fan.

An air purification device according to an example embodiment may include an air purification module configured to remove dust in air.

An air purification device according to an example embodiment may include an air sterilizing module disposed on the air purification module and configured to sterilize the air from which the dust has been removed by the air purification module.

### BRIEF DESCRIPTION OF THE DRAWINGS

The above and other aspects, features and advantages of certain embodiments of the present disclosure will be more apparent from the following detailed description, taken in conjunction with the accompanying drawings, in which:

FIG. 1 is a perspective view of an air purification module according to various embodiments;

FIG. 2 is a perspective view of the air purification module viewed from another angle according to various embodiments;

FIG. 3 is an exploded perspective view of the air purification module according to various embodiments;

FIG. 4 is an exploded perspective view of a main body frame, a filter assembly, an exhaust port grill, an exhaust port cover, and a support assembly, according to various embodiments;

FIG. 5 is a cross-sectional view of the air purification module according to various embodiments;

FIG. 6 is a perspective view of the support assembly according to various embodiments;

FIG. 7 is an exploded perspective view of the support assembly according to various embodiments;

FIG. 8 is an exploded perspective view illustrating coupling of the main body frame and a support frame, according to various embodiments;

FIG. 9 is an exploded perspective view illustrating mounting of a controller, according to various embodiments;

FIG. 10 is a perspective view of the controller mounted on a support frame, according to various embodiments;

FIG. 11 is a perspective view illustrating a connection wiring entering a wiring entrance portion of the main body frame, according to various embodiments;

FIG. 12 is an exploded perspective view of the controller according to various embodiments;

FIG. 13 is a diagram illustrating an arrangement in which the connection wiring is connected to a dust collection filter, according to various embodiments;

FIG. 14 is an exploded perspective view illustrating an arrangement in which the connection wiring is connected to a motor, according to various embodiments;

FIG. 15 is a perspective view illustrating coupling of the support frame and a frame lower cover, according to various embodiments;

FIG. 16 is a sectional perspective view illustrating coupling of the support frame and the frame lower cover, according to various embodiments;

FIG. 17 is a diagram illustrating a bottom surface of the air purification module according to various embodiments;

FIG. 18 is a cross-sectional view illustrating coupling of the main body frame, the support frame, and the frame lower cover, according to various embodiments;

FIG. 19 is a perspective view of a frame upper cover according to various embodiments;

FIG. 20 is a cross-sectional view illustrating coupling of the support frame, the frame upper cover, and the frame lower cover, according to various embodiments;

FIG. 21 is a cross-sectional view illustrating coupling of the frame upper cover and the main body frame, according to various embodiments;

FIG. 22 is a cross-sectional view illustrating a lower grill seating unit and a magnetic material, according to various embodiments;

FIG. 23 is a perspective view of an air purification device according to various embodiments;

FIG. 24 is a perspective view of the air purification device according to viewed from another angle according to various embodiments; and

FIG. 25 is a cross-sectional view of an air sterilization module according to various embodiments.

### DETAILED DESCRIPTION

Hereinafter, an air purification module and an air purification device according to various embodiments are described in greater detail with reference to the accompanying drawings. Throughout the drawings, like reference numerals denote like elements, and sizes of components in the drawings may be exaggerated for convenience of explanation and clarity. Terms such as first, second, etc. may be used for describing various components, but the components may not be limited to those terms. Such terms are used only for the purpose of distinguishing one element from another element.

As used herein, the singular forms “a,” “an,” and “the” are intended to include the plural forms as well, unless the context clearly indicates otherwise. Furthermore, when a part may “include” a certain element, unless specified otherwise, it may not be understood to exclude another element but may be construed to further include other elements. Furthermore, the size or thickness of each element illustrated in the drawings may be exaggerated for clarity of explanation.

The use of terms “a,” “an,” “the,” and similar referents in the context of describing the disclosure are to be construed to cover both the singular and the plural.

The steps of all methods described herein can be performed in any suitable order unless otherwise indicated herein or otherwise clearly contradicted by context. Furthermore, the use of any and all examples, or language (e.g., “such as”) provided herein, is intended merely to better illuminate the disclosure and does not pose a limitation on the scope of the disclosure unless otherwise claimed.

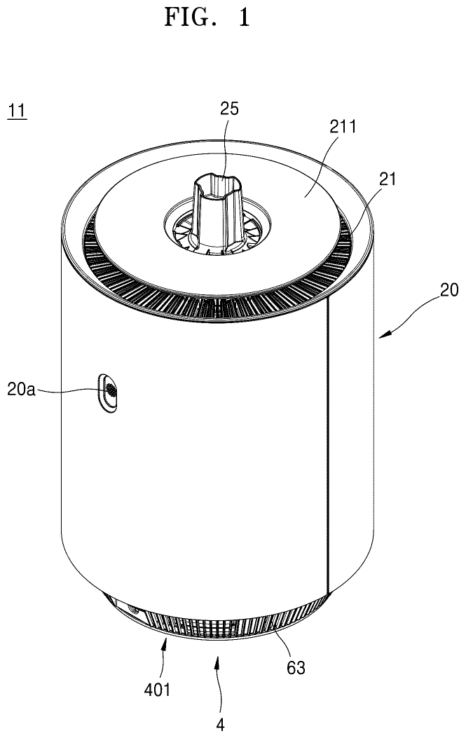

Terms, such as “an up/down direction,” “a height direction,” “a vertical direction,” etc., may refer, for example a Z direction based on the perspective view of an air purification device of FIG. 1, “a horizontal direction” may refer, for example, all directions according to an X-Y plane in an X direction or a Y direction based on the perspective view of FIG. 1, and the shape and the position of each component are not limited by the terms.

The terms, such as “a portion, a module, a member, or a block” used in the disclosure, may be implemented by software or hardware, according to various embodiments, a plurality of “portions, modules, members, or blocks” are implemented by one component, or one “portion, module, member, or block” may include a plurality of components.

Hereinafter, an air purification module and an air purification device according to various embodiments is described in greater detail with reference to the accompanying drawings.

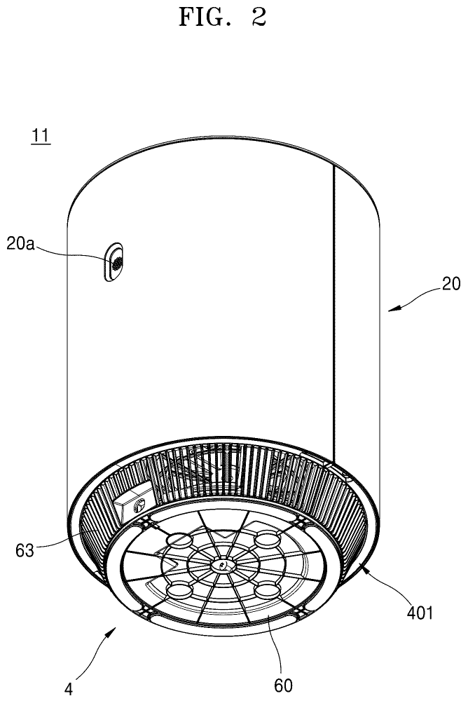

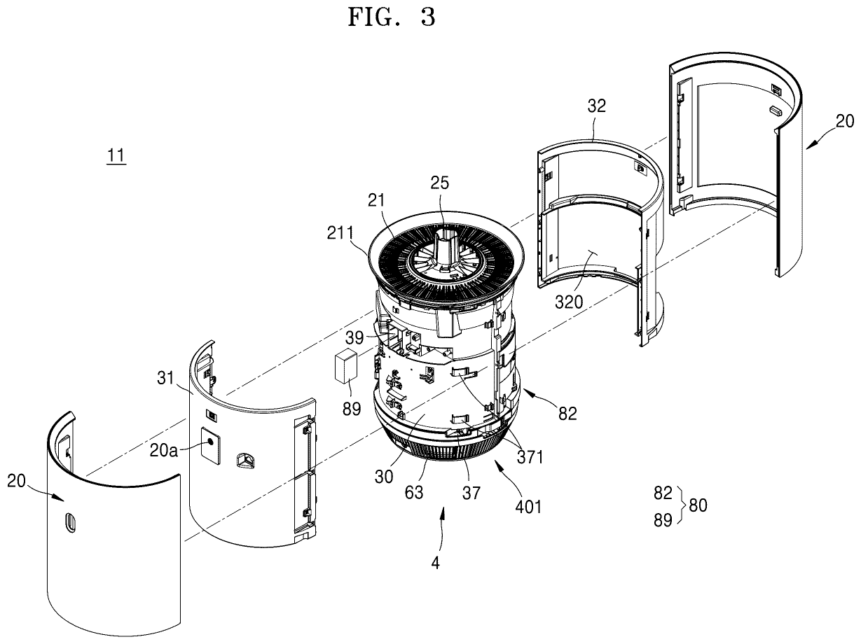

FIG. 1 is a perspective view of an air purification module according to various embodiments. FIG. 2 is a perspective view of the air purification module viewed from another angle according to various embodiments. FIG. 3 is an exploded perspective view of the air purification module according to various embodiments.

Referring to FIGS. 1, 2 and 3 (which may be referred to as FIGS. 1 to 3), an air purification module 11 according to an embodiment may include a housing 20, a main body frame 30, and a support assembly 4.

The housing 20 may accommodate the main body frame 30 and a fan 81 and provide a passage through which air may flow. The housing 20 may form the appearance of the air purification module 11. The housing 20 may include an exhaust port 21.

The fan 81 may be provided inside the housing 20. The fan 81 may be configured to generate airflow inside the housing 20. The fan 81 may suction external air from an intake port 401 of the air purification module 11 and discharge internal air from the exhaust port 21.

The main body frame 30 may be provided inside the housing 20 and support the fan 81. The main body frame 30 may form a frame of the air purification module 11. The main body frame 30 may support the housing 20. The main body frame 30 may include a material which is more rigid than that of the housing 20.

The main body frame 30 may include a frame front cover 31 and a frame rear cover 32. The frame front cover 31 and the frame rear cover 32 may function to prevent or reduce members supported by the main body frame 30 from protruding to the outside even when the housing 20 is removed from the air purification module 11.

The frame rear cover 32 may include a frame cover opening 320. The frame cover opening 320 may have the size through which a filter assembly 82 to be described below may enter and exit. The frame front cover 31 may function to protect a connection wiring 71 to be described below. The frame front cover 31 may not expose a wiring entrance unit 37 to be described below to the outside. The frame front cover 31 may protect a sensor 89 to be described below. The frame front cover 31 and the frame rear cover 32 may be integrated with the housing 20.

The exhaust port 21 may be provided in an upper portion of the main body frame 30. The exhaust port 21 may discharge the internal air of the air purification module 11 to the outside. The exhaust port 21 may include an exhaust port grill 210. The main body frame 30 may include an exhaust port cover 211. The exhaust port 21 may be surrounded by the exhaust port cover 211. The exhaust port cover 211 may induce flow of discharged air.

The main body frame 30 may include the sensor 89 and a sensor mounting unit 39. The sensor 89 may be accommodated in the sensor mounting unit 39. The sensor 89 may be configured to sense state of air such as an indoor space in which the air purification module 11 is installed. The sensor 89 may sense outside air through a sensor hole 20a included in the housing 20.

The air purification module 11 according to an embodiment may include a filter assembly 82. The filter assembly 82 may filter particulates in the air. The filter assembly 82 may remove dust or microorganisms from the air sucked through the intake port 401. The filter assembly 82 may be supported by the main body frame 30.

The support assembly 4 may support the main body frame 30. The support assembly 4 may support the main body frame 30 from a lower portion of the main body frame 30. The support assembly 4 may include the intake port 401. The intake port 401 may suction external air of the air purification module 11 into the air purification module 11. The intake port 401 may have an annular shape. The intake port 401 may suction external air from any direction on the plane.

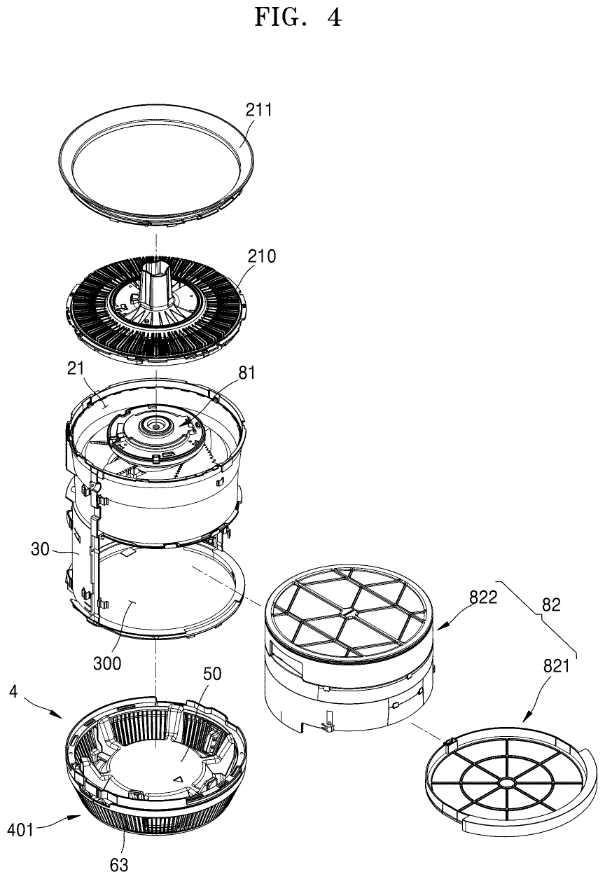

FIG. 4 is an exploded perspective view of a main body frame, a filter assembly, an exhaust port grill, an exhaust port cover, and a support assembly according to various embodiments.

Referring to FIGS. 3 and 4, the main body frame 30 according to an embodiment may include a main body frame opening 300. The filter assembly 82 may enter and exit through the main body frame opening 300.

The filter assembly 82 may include an electric dust collection filter 822 and a pre-filter 821. The pre-filter 821 may be provided to separate relatively large foreign substances from the air. The pre-filter 821 may be located upstream from an air flow direction compared to the electric dust collection filter 822. The pre-filter 821 may be inserted into a lower end of the electric dust collection filter 822. The electric dust collection filter 822 may be configured to collect dust using constant power. The electric dust collection filter 822 may form an electric field. Foreign substances such as dust in the air may be collected by the electric field formed by the electric dust collection filter 822. Organic substances such as bacteria and viruses may be decomposed by the electric field formed by the electric dust collection filter 822.

When the dust collection capacity of the pre-filter 821 or the electric dust collection filter 822 decreases, the filter assembly 82 of the air purification module 11 may need to be replaced. After removing the housing 20 of the air purification module 11 from the main body frame 30, the filter assembly 82 may be taken out and put back in again.

The fan 81 of the main body frame 30 according to an embodiment may be located downstream from the air flow direction compared to the filter assembly 82. When the fan 81 is located downstream from the air flow direction compared to the filter assembly 82, air from which particulates are removed from the filter assembly 82 may pass through the fan 81. The fan 81 may be located above the filter assembly 82. However, the position of the fan 81 is not limited thereto.

The exhaust port 21 may be provided in an upper portion of the fan 81. The exhaust port grill 210 may be disposed at the position of the exhaust port 21. The exhaust port grill 210 and the exhaust port cover 211 may be supported by the main body frame 30.

The support assembly 4 may be disposed in a lower portion of the main body frame 30 to support the main body frame 30. The support assembly 4 may support the weight of the air purification module 11 including the main body frame 30. The support assembly 4 may be coupled to the lower portion of the main body frame 30. An upper edge of the support assembly 4 may be connected to a lower edge of the main body frame 30.

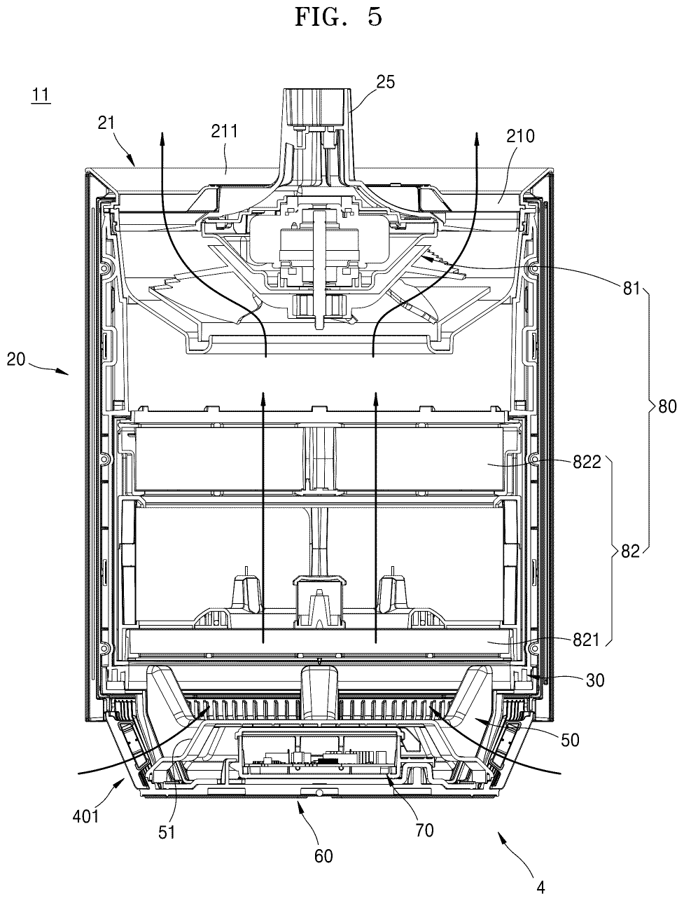

FIG. 5 is a cross-sectional view of the air purification module according to various embodiments.

Referring to FIG. 5, an air purification flow path may be formed inside the air purification module 11 according to an embodiment. Air outside the air purification module 11 may enter the air purification module 11 through the intake port 401 provided in the support assembly 4. The air entering the intake port 401 may move to the filter assembly 82 such that particulates may be removed. The air passing through the filter assembly 82 may be discharged to the outside of the air purification module 11 through the exhaust port 21 via the fan 81.

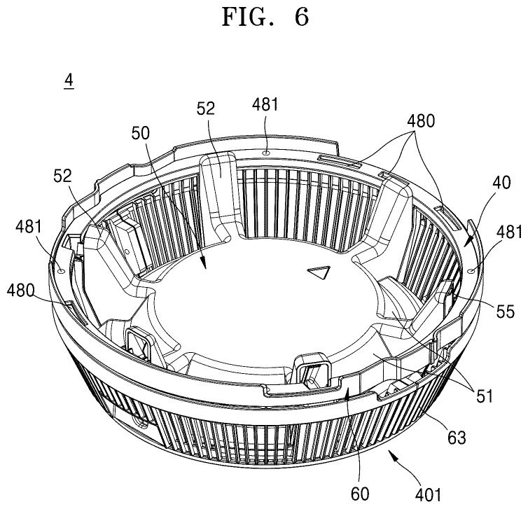

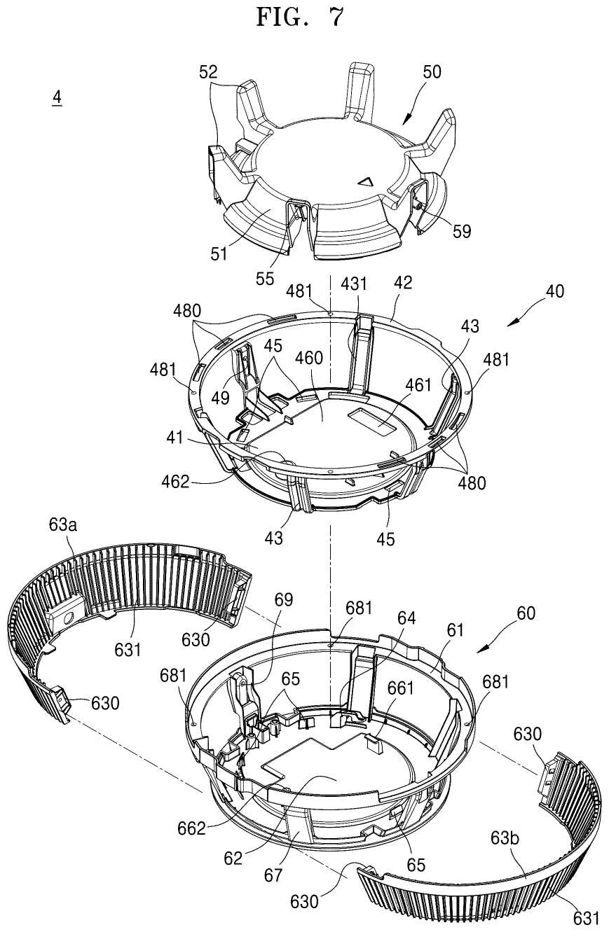

FIG. 6 is a perspective view of the support assembly according to various embodiments. FIG. 7 is an exploded perspective view of the support assembly according to various embodiments.

Referring to FIGS. 6 and 7, the support assembly 4 according to an embodiment may include a support frame 40, a frame upper cover 50, and a frame lower cover 60. The support frame 40 may support the weight of the air purification module 11. The support frame 40 may be connected to the main body frame 30 to support the load of the main body frame 30. The support frame 40 may seat a controller 70 to be described below.

The support frame 40 may include a base region 41 and an edge region 42.

The base region 41 may be a region in which the controller 70 is disposed. The base region 41 may have a flat plate shape. The base region 41 may have a disk shape. However, the shape of the base region 41 is not limited thereto.

The edge region 42 may be a region supporting the main body frame 30. The edge region 42 may be supported by the base region 41. The main body frame 30 may be supported by the base region 41 supporting the edge region 42. The edge region 42 may support the main body frame 30 so as to be positioned at a certain height from the base region 41. The edge region 42 may have an inner diameter and an outer diameter. The edge region 42 may have a band shape. The edge region 42 may have an annular shape. However, the shape of the edge region 42 is not limited thereto.

The edge region 42 may have an inner diameter longer than a circumferential length or an outer diameter of the base region 41. The edge region 42 may be located higher than the base region 41. The edge region 42 may be located at a certain height from the base region 41. When the edge region 42 has the inner diameter longer than the circumferential length of the base region 41, an inclined surface 51 to be described below may be formed. When the edge region 42 is located higher than the base region 41, the inclined surface 51 to be described below may be formed.

The intake port 401 may be formed between the edge region 42 and the base region 41. The intake port 401 may be arranged in a radial form from the center of the base region 41. The intake port 401 may suck air from a lower side of the air purification module 11 and transmit the air to an upper portion of the support assembly 4. The intake port 401 may have a shape that is inclined upward. The intake port 401 may have a shape that widens upward. However, the shape of the intake port 401 is not limited thereto.

The edge region 42 and the base region 41 may be connected to each other by a plurality of ribs 43. The plurality of ribs 43 may connect an end portion of the base region 41 to the base region 41. One end portion of the plurality of ribs 43 may be connected to the end portion of the base region 41. The plurality of ribs 43 may extend in a direction perpendicular to the base region 41, but may extend inclined in a direction away from the center of the base region 41 based on a plane formed by the base region 41. However, the shape of the plurality of ribs 43 is not limited thereto.

The plurality of ribs 43 may be disposed in an annular shape. The plurality of ribs 43 may be disposed at equal intervals. The plurality of ribs 43 may be disposed in four or more ribs. The base region 41, the edge region 42, and the plurality of ribs 43 may be one body. At least one of the base region 41, the edge region 42, and the plurality of ribs 43 may include a material which is more rigid than that of the frame upper cover 50. At least one of the base region 41, the edge region 42, and the plurality of ribs 43 may include a material which is more rigid than that of the frame lower cover 60. The material of the support frame 40 may be more rigid than the material of each of the frame upper cover 50 and the frame lower cover 60. The material of the support frame 40 may be more rigid than the material of each of the frame upper cover 50 or the frame lower cover 60.

The frame upper cover 50 may be disposed on the support frame 40. The frame upper cover 50 may be disposed on an upper portion of the base region 41. The frame upper cover 50 may include a material different from that of the support frame 40. The frame upper cover 50 may cover the support frame 40 from the top thereof. The frame upper cover 50 may protect the controller 70 to be described below. The frame upper cover 50 may guide the movement of air from the intake port 401 toward the fan 81.

The frame upper cover 50 may include the inclined surface 51. The inclined surface 51 may guide flow of air sucked through the intake port 401. The height of the inclined surface 51 may increase from the outer edge of the frame upper cover 50 to the center thereof. The inclined surface 51 may have a shape in which the diameter of the circumference decreases and is raised upward. The inclined surface 51 may have a substantially cut conical shape. However, the shape of the inclined surface 51 is not limited thereto.

The frame upper cover 50 may include a rib upper cover 52. The rib upper cover 52 may surround the plurality of ribs 43 from the top thereof. The rib upper cover 52 may be connected to the inclined surface 51. The rib upper cover 52 may protect the connection wiring 71 to be described below. The rib upper cover 52 may be raised higher than the edge region 42. In other words, an upper end of the rib upper cover 52 may be located higher than the edge region 42. An end portion of the rib upper cover 52 may be connected to the main body frame 30.

The frame lower cover 60 may cover the exterior of the support frame 40 in a lower portion of the support frame 40. The frame lower cover 60 may support the load of the support frame 40. The frame lower cover 60 may protect the support frame 40 so that the support frame 40 is not in direct contact with the ground.

The frame lower cover 60 may include a base cover 61 and an edge cover 62. The base cover 61 may support the base region 41. The base cover 61 may cover the base region 41. The base cover 61 may cover the outside of the base region 41 in a lower portion of the base region 41. The base cover 61 may support a load of the base region 41. The edge cover 62 may support the edge region 42. The edge cover 62 may cover the edge region 42.

The frame lower cover 60 may include a rib seating unit 67. The rib seating unit 67 may protect the plurality of ribs 43. The frame lower cover 60 may accommodate a lower grill 63 to be described below. The frame lower cover 60 may support the lower grill 63. The frame lower cover 60 may extend the intake port 401 formed by the support frame 40 to the outside of the support frame 40. The frame lower cover 60 may form the intake port 401.

The support assembly 4 according to an embodiment may include the lower grill 63. The lower grill 63 may surround the intake port 401. The lower grill 63 may be disposed on the intake port 401. Outside air may be sucked into the air purification module 11 through the lower grill 63. The lower grill 63 may form a plurality of slits 631 smaller than the intake port 401. The lower grill 63 may include a plurality of partial grills.

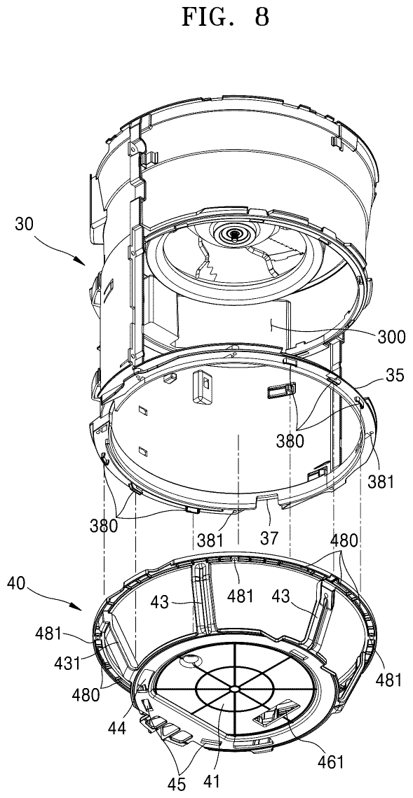

FIG. 8 is an exploded perspective view illustrating coupling of the main body frame and a support frame according to various embodiments.

Referring to FIG. 8, the main body frame 30 according to an embodiment may include a coupling latch 380. The coupling latch 380 may connect the main body frame 30 to the support frame 40. The coupling latch 380 may be located at a lower edge of the main body frame 30.

The support frame 40 according to an embodiment may include a locking groove 480. The edge region 42 may include the locking groove 480. The locking groove 480 may correspond to the coupling latch 380. The locking groove 480 may connect the main body frame 30 to the support frame 40. The coupling latch 380 and the locking groove 480 may respectively include a plurality of coupling latches and a plurality of locking grooves. The coupling latch 380 may include a protrusion. The coupling latch 380 may include the protrusion bent in a circumferential direction of the lower edge of the main body frame 30. The main body frame 30 and the support frame 40 may be fixed by engaging and turning the coupling latch 380 and the locking groove 480 in the circumferential direction of the main body frame 30. However, a method of fixing the main body frame 30 and the support frame 40 is not limited thereto. For example, the base region 41 of the support frame 40 may include the coupling latch 380, and the lower edge of the main body frame 30 may include the locking groove 480.

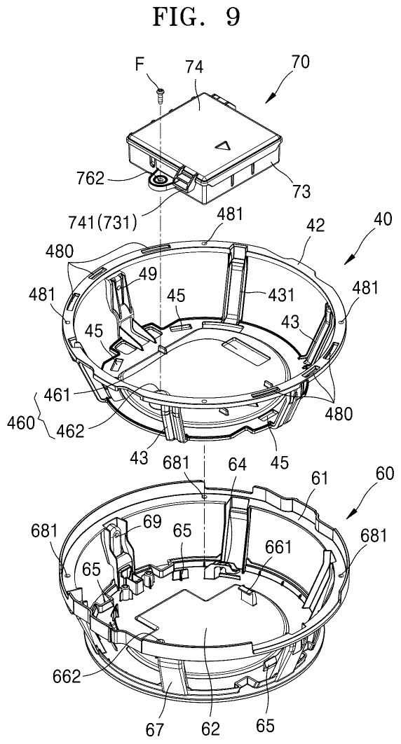

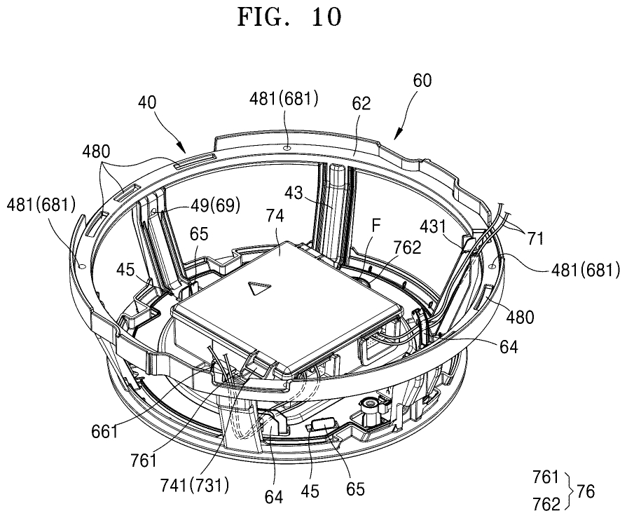

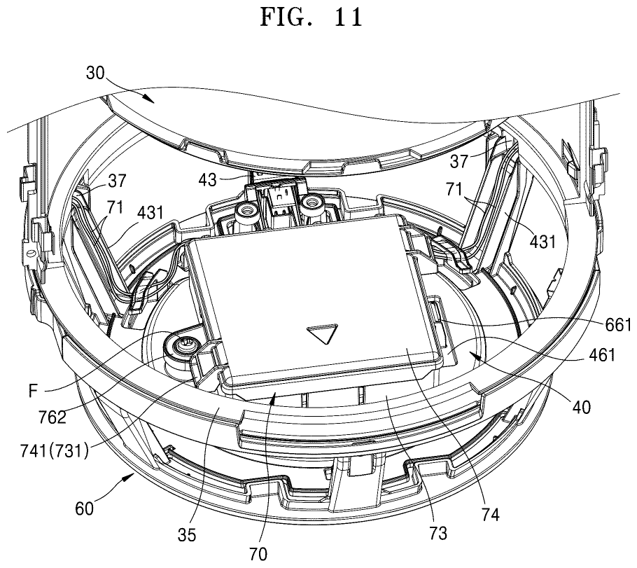

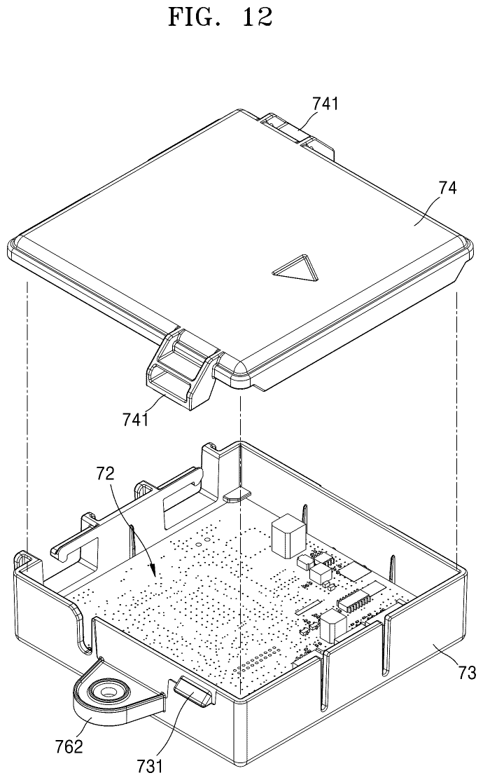

FIG. 9 is an exploded perspective view illustrating mounting of a controller according to various embodiments. FIG. 10 is a perspective view of the controller mounted on a support frame according to various embodiments. FIG. 11 is a perspective view illustrating a connection wiring entering a wiring entrance portion of the main body frame according to various embodiments. FIG. 12 is an exploded perspective view of the controller according to various embodiments.

Referring to FIGS. 3, 5, 9, 10, 11 and 12, the air purification module 11 according to an embodiment may include the controller 70. The controller 70 may include a control substrate 72. The control substrate 72 may control an electronic device module 80. The electronic device module 80 may include the fan 81, the filter assembly 82, and the sensor 89. The controller 70 may control the fan 81. The control substrate 72 may be electrically connected to the electronic device module 80. The control substrate 72 may be electrically connected to the fan 81, the filter assembly 82, and the sensor 89.

The controller 70 may include a control lower housing 73 and a control upper housing 74. The control substrate 72 of the controller 70 may be accommodated in the control lower housing 73. The control upper housing 74 may be coupled to the control lower housing 73. The control upper housing 74 may include a control upper housing catch 741. The control lower housing 73 may include a locking protrusion 731. The control upper housing catch 741 may be fixed to the locking protrusion 731. However, the coupling relationship between the control lower housing 73 and the control upper housing 74 is not limited thereto, and the control lower housing 73 and the control upper housing 74 may be one body.

The controller 70 may include a controller fixing member 76. The controller fixing member 76 may fix the controller 70 to the support assembly 4. The controller fixing member 76 may include a hook locking unit 761 and a first fixture insertion unit 762. The hook locking unit 761 may have a protrusion shape. The first fixture insertion unit 762 may include a screw-coupling hole.

The support frame 40 may include a control seating unit 460. The control seating unit 460 may fix the controller 70 to the support assembly 4. The control seating unit 460 may include a coupling hook penetration unit 461 and a control fixing seating unit 462. The coupling hook penetration unit 461 may have a penetration shape provided in the base region 41. The control fixing seating unit 462 may be a region including the screw-coupling hole protruding from the base region 41.

The frame lower cover 60 may include a control coupling hook 661 and a control coupling unit 662. When the support frame 40 and the frame lower cover 60 are coupled to each other, the control coupling hook 661 may penetrate the coupling hook penetration unit 461 and be raised upward from the base region 41. The control coupling unit 662 may include a screw-coupling hole. The screw-coupling holes of the control coupling unit 662, the control fixing seating unit 462, and the first fixture insertion unit 762 may be concentrically arranged.

The controller 70 may be fixed to the support assembly 4. The hook locking unit 761 of the controller 70 may be fixed by the control coupling hook 661 penetrating the coupling hook penetration unit 461. A fixing member F may fix the first fixture insertion unit 762, the control fixing seating unit 462, and the control coupling unit 662.

The controller 70 according to an embodiment may include the connection wiring 71. The connection wiring 71 may electrically connect the controller 70 to the electronic device module 80. The connection wiring 71 may electrically connect the controller 70 to the fan 81. The connection wiring 71 may include a plurality of connection wirings. The connection wiring 71 may be fixed to the wiring fixing hook 64.

The connection wiring 71 may be accommodated by the support assembly 4. The connection wiring 71 may extend in a direction of the main body frame 30. At least one of the plurality of ribs 43 may be a wiring cover rib 431. The wiring cover rib 431 may cover the connection wiring 71. The wiring cover rib 431 may protect the connection wiring 71. The connection wiring 71 may be accommodated in the wiring cover rib 431 and extend in the direction of the main body frame 30.

Referring to FIGS. 6, 7, 8, 9, 10 and 11, the frame upper cover 50 may cover an upper portion of the connection wiring 71 in the support assembly 4. The connection wiring 71 may be accommodated by the support frame 40 and the frame upper cover 50 in the base region 41 within the support assembly 4. The connection wiring 71 may be accommodated by the wiring cover rib 431 and the rib upper cover 52 when directed to the main body frame 30 along the plurality of ribs 43. The connection wiring 71 may pass between the plurality of ribs 43 and the rib upper cover 52. However, a method in which the connection wiring 71 is accommodated is not limited thereto. The connection wiring 71 may be accommodated by the support frame 40 and the frame lower cover 60. When the connection wiring 71 is directed to the main body frame 30, the connection wiring 71 may be accommodated by the wiring cover rib 431 and the rib seating unit 67. The connection wiring 71 may pass between the plurality of ribs 43 and the rib upper cover 52.

Referring to FIGS. 3, 8, and 11, the main body frame 30 according to an embodiment may include a wiring entrance unit 37 and a wiring fixing unit 371. The connection wiring 71 may enter toward an outer circumferential surface of the main body frame 30 through the wiring entrance unit 37. The connection wiring 71 may be fixed to the outer circumferential surface of the main body frame 30 by the wiring fixing unit 371. The wiring entrance unit 37 and the wiring fixing unit 371 may include a plurality of wiring entrance units and a plurality of wiring fixing units. The frame front cover 31 may protect the connection wiring 71. However, a path along which the connection wiring 71 heads toward the main body frame 30 is not limited thereto. For example, the connection wiring 71 may be connected to the top through an inner circumferential surface of the main body frame 30. For example, the connection wiring 71 may be connected to the electronic device module 80 through the inner circumferential surface of the main body frame 30.

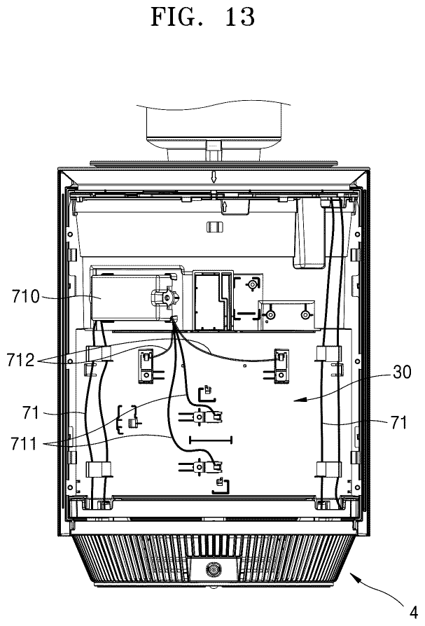

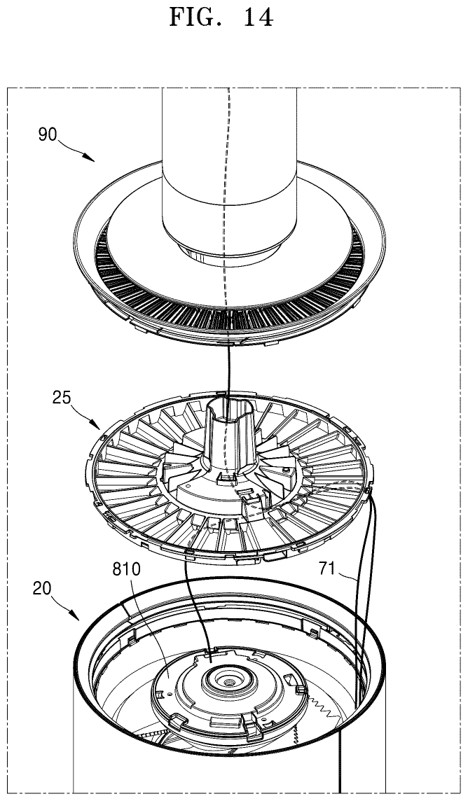

FIG. 13 is a diagram illustrating an arrangement in which the connection wiring is connected to a dust collection filter according to various embodiments. FIG. 14 is a an exploded perspective view illustrating an arrangement in which the connection wiring is connected to a motor according to various embodiments.

Referring to FIGS. 4, 11, 13, and 14, the connection wiring 71 according to an embodiment may enter the outer circumferential surface of the main body frame 30 through the wiring entrance unit 37 and be connected along the outer circumferential surface of the main body frame 30.

A part of the connection wiring 71 may be connected to the dust collection controller 710. The dust collection controller 710 may control the filter assembly 82. The dust collection controller 710 may provide a first dust collection wiring 711 connected to the pre-filter 821. The dust collection controller 710 may provide a second dust collection wiring 712 connected to the electric dust collection filter 822.

A part of the connection wiring 71 may be directed to an upper end of the main body frame 30 of an air purifier.

A part of the connection wiring 71 may be connected to a fan motor 810 of the fan 81.

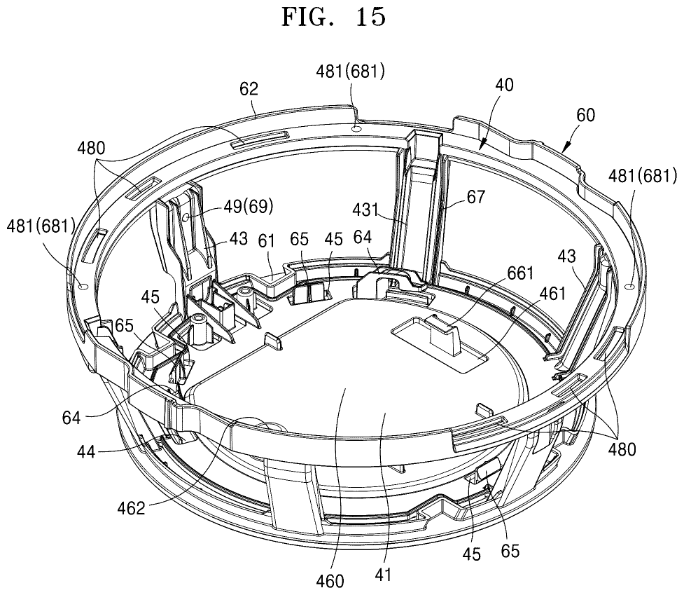

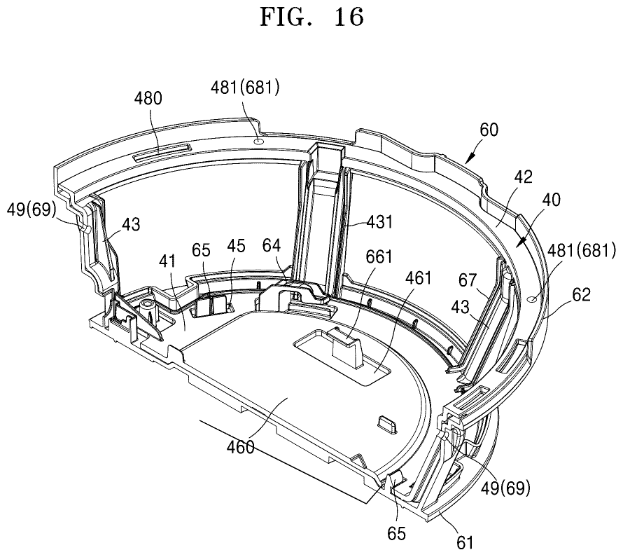

A part of the connection wiring 71 may be connected to an air sterilization module 90 through an air purification grill 25. FIG. 15 is a perspective view for explaining coupling of the support frame and a frame lower cover according to an embodiment. FIG. 16 is a cross-sectional perspective view for explaining coupling of the support frame and the frame lower cover according to an embodiment.

Referring to FIGS. 15 and 16, the frame lower cover 60 according to an embodiment may include a frame coupling hook 65. The support frame 40 may include a lower hook locking groove 45 corresponding to the frame coupling hook 65. The base region 41 may include the lower hook locking groove 45 corresponding to the frame coupling hook 65. The frame coupling hook 65 and the lower hook locking groove 45 may couple the support frame 40 to the frame lower cover 60. The frame coupling hook 65 may include a hook structure that may be locked in the lower hook locking groove 45. The frame coupling hook 65 may include a plurality of frame coupling hooks.

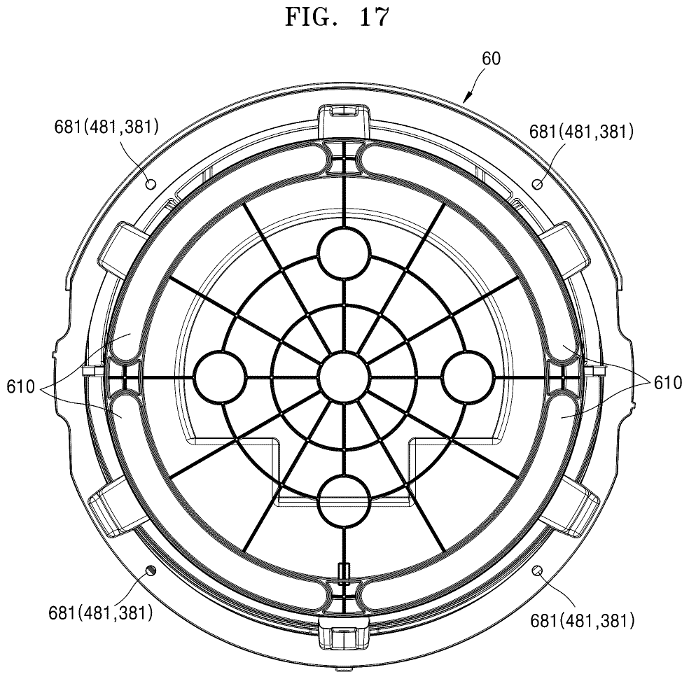

FIG. 17 is a diagram illustrating a bottom surface of the air purification module according to various embodiments.

Referring to FIG. 17, the air purification module 11 according to an embodiment may include an anti-slip pad 610. The frame lower cover 60 may include the anti-slip pad 610. The base cover 61 may include the anti-slip pad 610. The anti-slip pad 610 may prevent or suppress the air purification module 11 from slipping on the floor.

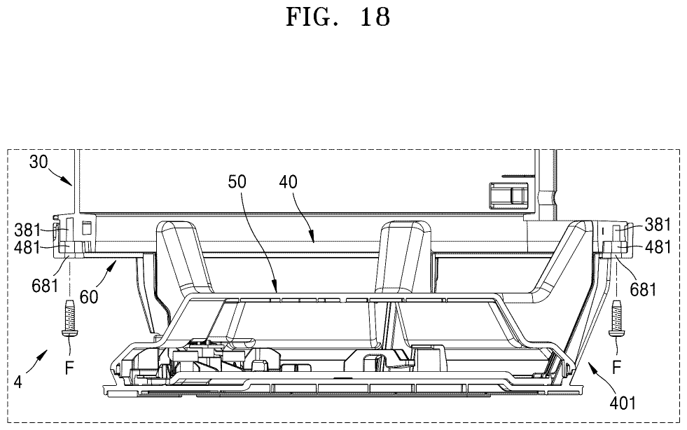

FIG. 18 is a cross-sectional view illustrating coupling of the main body frame, the support frame, and the frame lower cover according to various embodiments.

Referring to FIGS. 15, 16, 17 and 18, the main body frame 30 according to an embodiment may include a support coupling unit 381. The support coupling unit 381 may couple the main body frame 30 to the support assembly 4. The support frame 40 according to an embodiment may include a body coupling unit 481. The body coupling unit 481 may couple the support frame 40 to the body coupling unit 481. The frame lower cover 60 according to an embodiment may include a second fixture insertion unit 681. The second fixture insertion unit 681 may correspond to the support coupling unit 381 and the second fixture insertion unit 681. At least two of the second fixture insertion unit 681, the body coupling unit 481, and the support coupling unit 381 may have the same axis. The main body frame 30, the support frame 40, and the frame lower cover 60 may be fixed by inserting the fixing member F into the second fixture insertion unit 681. However, a method of fixing the main body frame 30, the support frame 40, and the frame lower cover 60 is not limited thereto. For example, the main body frame 30, the support frame 40, and the frame lower cover 60 may be fixed by inserting the fixing member F into a direction of the main body frame 30.

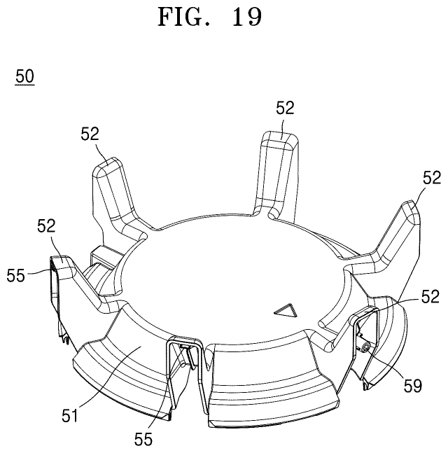

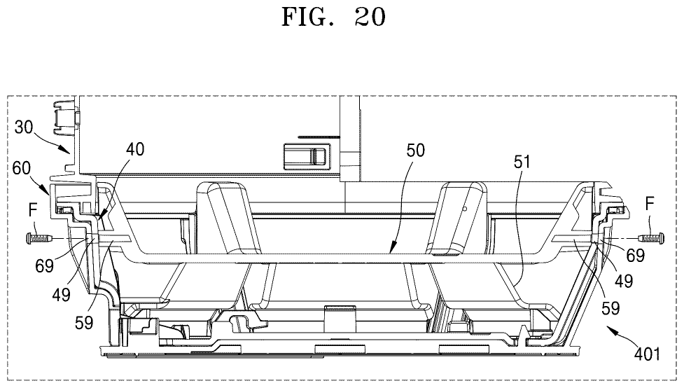

FIG. 19 is a perspective view of the frame upper cover according to various embodiments. FIG. 20 is a cross-sectional view illustrating coupling of the support frame, the frame upper cover, and the frame lower cover according to various embodiments.

Referring to FIGS. 9, 11, 12, 15, 16, 19, and 20, the support frame 40 according to an embodiment may include a cover coupling unit 49. The cover coupling unit 49 may couple the frame upper cover 50 to the support frame 40. The frame upper cover 50 according to an embodiment may include a frame coupling unit 59. The frame coupling unit 59 may couple the support frame 40 to the frame upper cover 50. The frame lower cover 60 according to an embodiment may include a third fixture insertion unit 69. The frame lower cover 60 may correspond to the cover coupling unit 49 and the frame coupling unit 59. At least two of the third fixture insertion unit 69, the frame coupling unit 59, and the cover coupling unit 49 may have the same axis. The support frame 40, the frame lower cover 60, and the support frame 40 may be fixed by inserting the fixing member F into the third fixture insertion unit 69. However, a method of fixing the support frame 40, the frame upper cover 50, and the frame lower cover 60 is not limited thereto. For example, the support frame 40, the frame upper cover 50, and the frame lower cover 60 may be fixed by inserting the fixing member F into a direction of the frame upper cover 50.

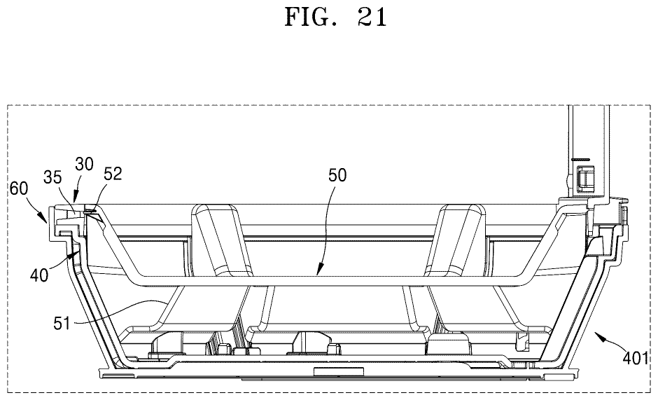

FIG. 21 is a cross-sectional view illustrating coupling of the frame upper cover and the main body frame according to various embodiments.

Referring to FIGS. 19 and 21, the frame upper cover 50 according to an embodiment may include a main body coupling hook 55. The main body coupling hook 55 may couple the frame upper cover 50 to the main body frame 30. The main body frame 30 according to an embodiment may include a coupling groove 35. The coupling groove 35 may correspond to the main body coupling hook 55. The coupling groove 35 may couple the frame upper cover 50 to the main body frame 30. The coupling groove 35 and the main body coupling hook 55 may respectively include a plurality of coupling grooves and a plurality of main body coupling hooks. However, a method of coupling the main body frame 30 to the frame upper cover 50 is not limited thereto. For example, the main body frame 30 may include a hook, and the frame upper cover 50 may include a groove corresponding thereto.

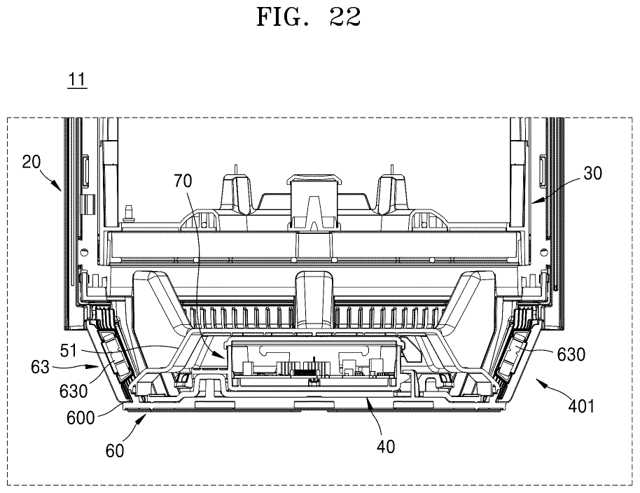

FIG. 22 is a cross-sectional view for explaining a lower grill seating unit and a magnetic material according to various embodiments.

The frame lower cover 60 according to an embodiment may include a lower grill seating unit 600. An end portion of the lower grill 63 may be seated on the lower grill seating unit 600. The lower grill seating unit 600 may guide seating of the frame lower cover 60. The lower grill seating unit 600 may be in a lower portion of the frame lower cover 60. The lower grill seating unit 600 may have a concave shape in accordance with the shape of the end portion of the lower grill 63. However, the position and shape of the frame lower cover 60 are not limited thereto.

Referring to FIGS. 9 and 22, the lower grill 63 according to an embodiment may include a magnetic body 630. The magnetic body 630 may include a material that is magnetic or reacts to a magnetic force. The lower grill 63 may include a plurality of partial grills. The magnetic body 630 may couple a plurality of partial grills of the lower grill 63. The lower grill 63 may include a first partial grill 63a and a second partial grill 63b. The magnetic body 630 may be disposed at end portions of the first partial grill 63a and the second partial grill 63b. However, the number or shapes of the partial grills of the lower grill 63 is not limited thereto. For example, three or more partial grills may form the lower grill 63.

The air purification module 11 according to an embodiment may be an air purification device 1. The air purification device 1 may include the air purification module 11.

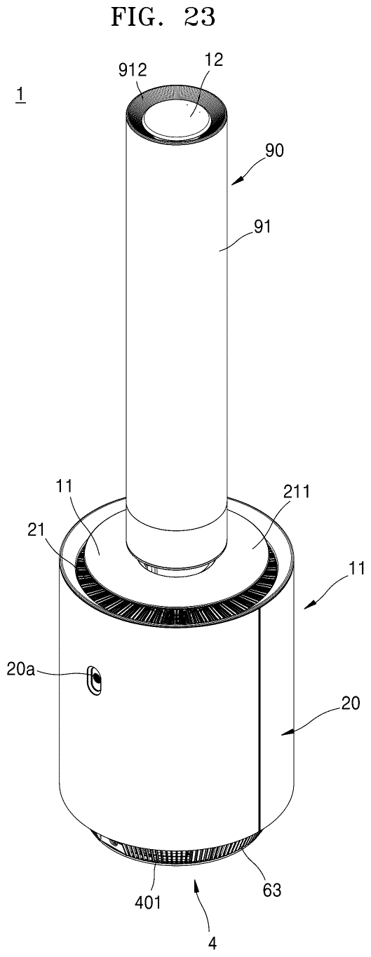

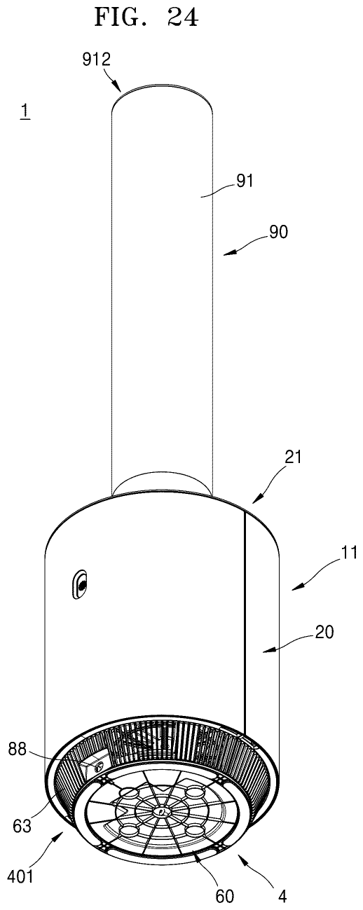

FIG. 23 is a perspective view of an air purification device according to various embodiments. FIG. 24 is a perspective view of the air purification device viewed from another angle according to various embodiments.

Referring to FIGS. 23 to 24, the air purification device 1 according to an embodiment may include an air sterilization module 90. The air sterilization module 90 may be disposed on the air purification module 11. The air purification module 11 may support the air sterilization module 90. The support assembly 4 may support the air purification device 1. The support assembly 4 may support the weight of each of the air purification module 11 and the air sterilization module 90.

The support assembly 4 according to an embodiment may include a power supply unit 88. The power supply unit 88 may supply power to the controller 70. The power supply unit 88 may supply power to the electronic device module 80.

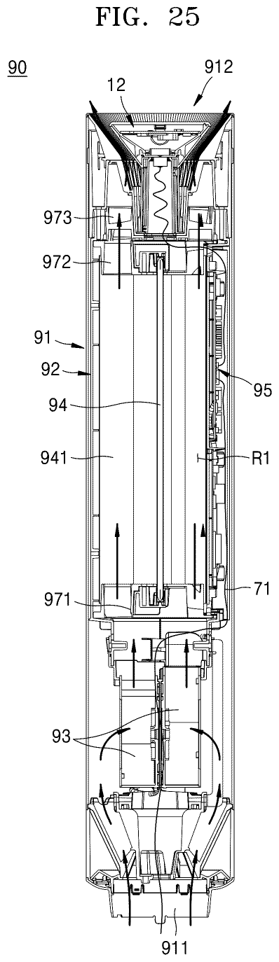

FIG. 25 is a cross-sectional view of an air sterilization module according to various embodiments.

Referring to FIGS. 23, 24 and 25, the air sterilization module 90 according to an embodiment may include a sterilization module housing 91 and a sterilization fan 93. The sterilization fan 93 may be configured to generate airflow inside the air sterilization module 90. The sterilization module housing 91 may accommodate the sterilization fan 93 and provide a passage through which air may flow. The sterilization module housing 91 may form the appearance of the air sterilization module 90. The sterilization module housing 91 may include a sterilization intake port 911 and a sterilization exhaust port 912. The sterilization intake port 911 may suction air into the air sterilization module 90. The sterilization exhaust port 912 may discharge air from the inside of the sterilization module housing 91 to the outside of the sterilization module housing 91. The sterilization intake port 911 may be in a lower portion of the air sterilization module 90. The sterilization exhaust port 912 may be in an upper portion of the air sterilization module 90. However, the locations of the sterilization intake port 911 and the sterilization exhaust port 912 are not limited thereto.

The air sterilization module 90 according to an embodiment may suck, from the sterilization intake port 911, at least part of the air discharged from the exhaust port 21 by the air purification module 11. The air purification module 11 according to an embodiment may move at least part of the air from the exhaust port 21 to the air sterilization module 90.

The air sterilization module 90 according to an embodiment may include a sterilization module frame 92. The sterilization module frame 92 may form a frame of the air sterilization module 90. The sterilization module frame 92 may support the sterilization module housing 91. The sterilization module frame 92 may include a material which is more rigid than that of the sterilization module housing 91. The electronic device module 80 may include a sterilization fan 93 and an ultraviolet irradiation module 94. The sterilization module frame 92 may support the electronic device module 80.

The sterilization module frame 92 may be connected to the main body frame 30 of the air purification module 11. The main body frame 30 may support the sterilization module frame 92.

The air sterilization module 90 according to an embodiment may include the ultraviolet irradiation module 94. The ultraviolet irradiation module 94 may sterilize microorganisms in the air by irradiating ultraviolet rays. An irradiation region RI may be a region to which the ultraviolet rays emitted by the ultraviolet irradiation module 94 is irradiated. Air flowing inside the air sterilization module 90 may be sterilized while passing through the irradiation region RI.

The air sterilization module 90 according to an embodiment may include a reflective cover 941 and a light blocking member 97. The reflection cover 941 may surround the ultraviolet irradiation module 94. The reflective cover 941 may internally reflect the ultraviolet rays irradiated by the ultraviolet irradiation module 94. The light blocking member 97 may be located in upper and lower portions of the ultraviolet irradiation module 94. The light blocking member 97 may include a first light blocking member 971, a second light blocking member 972, and a third light blocking member 973. The first light blocking member 971 may prevent or block ultraviolet rays from leaking out of the sterilization intake port 911. The second light blocking member 972 and the third light blocking member 973 may prevent or block ultraviolet light from leaking out of the sterilization exhaust port 912.

The air sterilization module 90 according to an embodiment may include a sterilization control substrate 95. The sterilization control substrate 95 may control the ultraviolet irradiation module 94. The sterilization control substrate 95 may control the sterilization fan 93. The controller 70 of the air purification device 1 may control the sterilization control substrate 95.

The connection wiring 71 according to an embodiment may be connected to the sterilization control substrate 95. The sterilization control substrate 95 may be electrically connected to the controller 70 (see FIGS. 11 and 12).

The air sterilization module 90 according to an embodiment may include a display module 12. The display module 12 may be electrically connected to the sterilization control substrate 95. The display module 12 may provide information about the operation of the air purification device 1.

The air purification module 11 according to an embodiment may provide the support assembly 4 capable of supporting its own weight.

The support assembly 4 according to an embodiment may suck air in all directions on a plane supporting the air purification module 11.

The support assembly 4 according to an embodiment may provide the intake port 401 capable of sucking outside air in any direction on the plane.

The support assembly 4 according to an embodiment may provide the inclined surface 51 capable of guiding air sucked in an upward direction.

The support assembly 4 according to an embodiment may accommodate the controller 70 in a lower portion thereof, thereby sufficiently securing an air purification space inside the housing 20.

The support assembly 4 according to an embodiment may provide the intake port 401 that does not interfere with the flow of suctioned air while firmly supporting the air purification module 11.

An air purification module according to an example embodiment may include: a housing, a fan provided inside the housing and configured to generate a flow inside the housing, a main body frame provided inside the housing and configured to support the fan, a support assembly including a support disposed in a lower portion of the main body frame and configured to support the main body frame and configured to provide an intake port through which air is taken into the inside of the housing, and a controller, comprising circuitry, disposed in the support assembly and configured to control the fan, wherein the support assembly includes a base region in which the controller is disposed, an edge region configured to support the main body frame to be positioned at a specified height from the base region, and a support frame including a plurality of ribs configured to connect the edge region and the base region so that the intake port is provided between the base region and the edge region, and a frame upper cover including a material different from a material of the support frame, disposed on an upper portion of the base region, configured to protect the controller, and to guide a movement of air from the intake port toward the fan.

The intake port may be arranged in a radial shape from the center of the base region.

The frame upper cover may include an inclined surface having a height increasing from an outer edge of the frame upper cover to the center thereof.

The frame upper cover may include a rib upper cover configured to surround the plurality of ribs from the top thereof.

The air purification module may further include a connecting wiring configured to electrically connect the controller and the fan, and the connecting wiring may be accommodated by the support assembly.

The connecting wiring may be disposed to pass between the plurality of ribs and the rib upper cover.

The support assembly may include a frame lower cover configured to cover an exterior of the support frame in a lower portion of the support frame.

The support assembly may include a lower grill configured to surround the intake port and including a plurality of slits smaller than the intake port.

A material of the support frame may be more rigid than a material of the frame upper cover.

At least one of the support frame and the main body frame may include a coupling latch, and at least one of the support frame and the main body frame may include a locking groove corresponding to the coupling latch.

The frame lower cover may include a frame coupling hook, and the support frame may include a lower hook locking groove corresponding to the frame coupling hook.

The frame upper cover may include a main body coupling hook, and the main body frame include a coupling groove corresponding to the main body coupling hook.

An air purification device according to an example embodiment includes: an air purification module configured to remove dust in air, and an air sterilizing module including sterilization circuitry disposed on the air purification module and configured to sterilize the air from which the dust has been removed by the air purification module, wherein the air purification module includes: a housing, a fan provided inside the housing and configured to generate a flow inside the housing, a main body frame provided inside the housing and configured to support the fan, a support assembly including a support disposed in a lower portion of the main body frame and configured to support the main body frame and to provide an intake port through which air is taken into the inside of the housing, and a controller, comprising circuitry, disposed in the support assembly and configured to control the fan, wherein the support assembly includes a base region in which the controller is disposed, an edge region configured to support the main body frame to be positioned at a specified height from the base region, and a support frame including a plurality of ribs configured to connect the edge region and the base region so that the intake port is formed between the base region and the edge region, and a frame upper cover having a material different from a material of the support frame, disposed on an upper portion of the base region, configured to protect the controller, and guide a movement of air from the intake port toward the fan.

The intake port may be arranged in a radial shape from the center of the base region.

The frame upper cover may include an inclined surface having a height increasing from an outer edge of the frame upper cover to the center thereof.

While the disclosure has been illustrated and described with reference to various example embodiments, it will be understood that the various example embodiments are intended to be illustrative, not limiting. It will be further understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

What is claimed is:

1. An air purification module comprising:
a housing;
a fan provided inside the housing and configured to generate a flow inside the housing;
a main body frame provided inside the housing and configured to support the fan;
a support assembly including a support disposed in a lower portion of the main body frame and configured to support the main body frame and to provide an intake port through which air is taken into the inside of the housing; and
a controller, comprising circuitry, disposed in the support assembly and configured to control the fan,
wherein the support assembly comprises
a base region in which the controller is disposed, an edge region configured to support the main body frame to be positioned at a specified height from the base region, and a support frame comprising a plurality of ribs configured to connect the edge region and the base region so that the intake port is provided between the base region and the edge region, and
a frame upper cover including a material different from a material of the support frame, disposed on an upper portion of the base region, and configured to protect the controller and to guide a movement of air from the intake port toward the fan.

2. The air purification module of claim 1, wherein:
the intake port is arranged in a radial shape from the center of the base region.

3. The air purification module of claim 1, wherein
the frame upper cover includes:
an inclined surface having a height increasing from an outer edge of the frame upper cover to a center of the frame upper cover.

4. The air purification module of claim 1, wherein:
the frame upper cover includes:
a rib upper cover configured to surround the plurality of ribs from a top of the frame upper cover.

5. The air purification module of claim 4, further comprising:
a connecting wiring configured to electrically connect between the controller and the fan, and
wherein the connecting wiring is accommodated by the support assembly.

6. The air purification module of claim 5, wherein
the connecting wiring is disposed to
pass between the plurality of ribs and the rib upper cover.

7. The air purification module of claim 1, wherein:
the support assembly includes:
a frame lower cover configured to cover an exterior of the support frame in a lower portion of the support frame.

8. The air purification module of claim 1, wherein
the support assembly includes:
a lower grill configured to surround the intake port and form a plurality of slits smaller than the intake port.

9. The air purification module of claim 1, wherein:
a material of the support frame is
more rigid than a material of the frame upper cover.

10. The air purification module of claim 1, wherein:
at least one of the support frame and the main body frame includes a coupling latch, and
at least one of the support frame and the main body frame includes a locking groove corresponding to the coupling latch.

11. The air purification module of claim 7, wherein:
the frame lower cover includes a frame coupling hook, and
the support frame includes a lower hook locking groove corresponding to the frame coupling hook.

12. The air purification module of claim 1, wherein:
the frame upper cover includes a main body coupling hook, and
the main body frame includes a coupling groove corresponding to the main body coupling hook.

13. An air purification device comprising:
an air purification module configured to remove dust in air; and
an air sterilizing module comprising sterilization circuitry disposed on the air purification module and configured to sterilize the air from which the dust has been removed by the air purification module,
wherein the air purification module comprises:
a housing, and a fan provided inside the housing and configured to generate a flow inside the housing;
a main body frame provided inside the housing and configured to support the fan;
a support assembly including a support disposed in a lower portion of the main body frame and configured to support the main body frame and to provide an intake port through which air is taken into the inside of the housing; and
a controller comprising circuitry disposed in the support assembly and configured to control the fan, wherein:
the support assembly comprises:
a base region in which the controller is disposed, an edge region configured to support the main body frame to be positioned at a specified height from the base region, and a support frame comprising a plurality of ribs configured to connect the edge region and the base region so that the intake port is formed between the base region and the edge region, and
a frame upper cover comprising a material different from a material of the support frame, disposed on an upper portion of the base region, and configured to protect the controller and to guide a movement of air from the intake port toward the fan.

14. The air purification device of claim 13, wherein:
the intake port is arranged in a radial shape from the center of the base region.

15. The air purification device of claim 13, wherein
the frame upper cover includes:
an inclined surface having a height increasing from an outer edge of the frame upper cover to a center of the frame upper cover.
